(19) United States
(12) Patent Application Publication
(10) Pub. No.: US 2006/0177392 A1
(43) Pub. Date: Aug. 10, 2006

(54) Oil-based composition for acne

(76) Inventor: William Walden, Hillsboro, OR (US)

Correspondence Address: William Otis Walden, 20348 NW Colonnade Drive, Hillsboro, OR, 97124, US

(21) Appl. No.: 11/293,692

(22) Filed: Dec. 5, 2005

Related U.S. Application Data
(60) Provisional application No. 60/651,263, filed on Feb. 10, 2005.

(51) Int. Cl. A61K 31/203; A61K 31/22; A61K 8/36; A61K 31/07

(52) U.S. Cl. 424/59; 424/401; 514/559; 514/725; 514/546

(57) ABSTRACT

An oil-based topical composition for use on the skin containing at least one compound from the class of retinoids, which are useful as medicinal agents, in an oleaginous solution composed substantially of non-ionic lipids, which are useful as vehicles for nonpolar compounds.

### CROSS-REFERENCE TO RELATED APPLICATIONS

This application is entitled to the benefit of Provision Patent Application Ser. No. 60/651,263 filed 2005 on the 10th of February.

### FEDERALLY SPONSORED RESEARCH/DEVELOPMENT

Not Applicable

### SEQUENCE LISTING

Not Applicable

### BACKGROUND—FIELD OF INVENTION

This invention relates to compositions and methodologies for topical application to mammalian skin, specifically to such applications which normalize sebaceous activity for the treatment of oily skin, acne, blackheads, acne scars, and hair loss.

### BACKGROUND—DESCRIPTION OF PRIOR ART

Acne vulgaris is a common disease which afflicts approximately 90% of all teenagers, and not uncommonly, affects men and women in their twenties or thirties, or may persist throughout adulthood. It is a condition of the human skin characterized by an excess flow of oil (sebum) from the sebaceous glands located within the pores of mammalian skin. The cells of these glands from which sebum originates, known as sebocytes, commonly occur in high concentration on the face, ears, retroauricular areas (e.g. behind the ears), chest, back, neck, and upper arms.

As a secretion of sebocytes, sebum production is regulated by endogenous hormones, particularly sex-hormones such as testosterone, thus varies in serum concentration between individuals. Sebum normally exits at the skin's surface through the hair follicle; however, an excessive quantity of sebum in this duct and/or on the skin may block its egress from the follicular opening. Such blockage causes hyperkeratinization of this lipid-laden product of sebaceous glands, whereby the mass thickens and solidifies to form a solid plug in the pore known as a “comedo.”

The comedo, commonly known as a “blackhead,” is the earliest manifestation and most fundamental lesion associated with acne. The solid keratinized sebum in comedones (plural of comedo) provides an excellent growth medium for anaerobic microorganisms, namely Propionibacterium acnes (P. acnes). P. acnes generate irritating free fatty acids which, in turn, prompts the immune system to react by forming papules, pustules, and cysts, and if these swell to rupture follicular walls, bacteria and microbially-derived irritants contaminate adjacent dermal tissue and initiate secondary infections. Depending upon the degree of inflammation, acne can result in slight skin irritation to pitting, scars, and disfiguration.

Topical keratolytic agents, i.e., benzoyl peroxide, have been employed in the treatment of acne to prevent the blocking of the follicular duct and/or act against bacteria. However, these agents can not address acne's root-cause, vis-à-vis sebum production, and even in acne's absence, the presence of excessive amounts of sebum on the skin results in an unattractive cosmetic condition known as “oily skin.” Persons with this skin type typically manifest a shiny complexion. To avoid such appearance, individuals may commonly cleanse, blot, apply oil absorbing powders to the skin, and/or remove skin oils with low molecular weight alcohols. But these techniques offer only temporary reprieve from oiliness, as removed sebum is quickly replaced by the copious and continuous output of overactive sebaceous glands, so for both cosmetic and dermatological reasons, there exists a need to reduce sebum synthesis by the sebaceous glands to both minimize the oily appearance of the skin and eliminate the source of numerous maladies, including acne, attributed to excessive sebum production.

Skin care compositions containing “retinoids” have become of great interest in recent years. Chemically, Vitamin A belongs to a group of compounds known as “retinoids,” consisting of four isoprenoid units joined in a head-to-tail manner. All retinoids may be formally derived from a monocyclic parent compound containing five carbon-carbon double bonds and a functional group at the end of the acyclic portion. As used herein, “retinoid” includes all natural and/or synthetic analogs of Vitamin A or retinol-like compounds which possess the biological activity of Vitamin A in the skin as well as the geometric isomers and stereoisomers of these compounds, such as retinoic acid, retinol (Vitamin A alcohol), retinal (Vitamin A aldehyde) and retinyl esters (e.g., retinyl acetate and retinyl palmitate).

Following discovery of the first vitamin in 1912, investigations would soon commence concerning the use of Vitamin A for treating skin conditions. Since, over 1,500 synthetic retinoids have been developed. Of these, isotretinoin (13-cis-retinoic acid), marketed by Roche Laboratories, Nutley, N.J., under the trademark “Accutane,” is among the most commonly used retinoids for treating acne.

Though exhibiting utility for a variety of skin disorders, all natural and synthetic retinoids pose significant health risks when taken internally. Sustained Vitamin A doses of over 50,000 IU may be toxic, and a single megadose of 1,500,000 IU can induce brain swelling, headache, drowsiness, vomiting, and/or death. Overdose may also cause itching, irritability, swelling, joint pain, weight loss, bleeding lips, hair loss, brittle nails, increased blood calcium, and cirrhosis of the liver. The typical dose ranging from 0.5 to 2 mg/kg body weight per 24 hours of Accutane is often teratogenic and commonly reported to precipitate human fetal abnormalities and spontaneous abortions.

As retinoids cannot penetrate the bloodstream via topical application, a more direct method for reducing sebum is desired. Well-known for the treatment of skin conditions, products containing retinoic acid are commercially available in various forms from the Dermatological Division of Ortho Pharmaceutical Corporation, and marketed under the trademark “Retin A.” Numerous creams, water-in-oil, and oil-in-water emulsions utilizing retinoic acid have been put forth by U.S. Pat. No. 5,837,270 to Burgess et al. (1998), U.S. Pat. No. 6,531,141 to Marvel (2003), and U.S. Pat. No. 5,670,547 to Milstein et al. (1997).

In addition, many topically administered compositions and methodologies employing natural retinoids have been proposed: U.S. Pat. No. 5,520,919 to Lerner (1996); U.S. Pat. No. 5,932,228 to Hall et al. (1999); U.S. Pat. No. to Liu et al 5,976,555 (1999); U.S. Pat. No. 6,150,403 to Biedermann et al. (2000); U.S. Pat. No. to Coury et al. (2003); and U.S. Pat. No. 6,645,510 to Boussouira et al. (2000). However, all of these topically administered compositions and methodologies are ineffective due to a self-imposed restriction that their compositions be non-greasy and so deemed aesthetically acceptable to consumers. While alcohol-based preparations tend to extract lipids from the skin surface, leading to dry skin and discontinuation of use, water-based formulations avoid the harsh effects of irritating organic solvents, but since active retinoids are not water-soluble, they cannot penetrate the horny outermost layer of the skin composed of closely-packed keratinized cells, known as the “stratum corneum,” and therefore cannot interact with sebocytes couched in the lowest dermal layers distal to the stratum corneum.

### SUMMARY

In accordance with the present invention, we disclose an oil-based composition to enhance the topical delivery of retinoids on mammalian skin for abating a wide range of disorders attributed to overactive sebaceous glands.

### DETAILED DESCRIPTION OF THE INVENTION—OVERVIEW

That a medicinal treatment is safe is more important than its efficacy. Because Vitamin A and its derivatives like Accutane are toxic in overdose, a topical methodology for reducing sebum output without causing significant adverse side-effects to the skin or other organs is preferred.

The limitations of other topical treatments have been discussed in the prior art portion of this application. Their attempts to create a functional vehicle fail via insistence that their formulas be non-greasy. Oil-based, the present invention violates their aesthetic requirement, as the ability to successfully treat diseases outweighs manner of application. Since most retinoids are water-insoluble, hydrophobic, lipid-soluble compounds, the nonpolar characteristic of the present invention provides an effective vehicle for allowing Vitamin A compounds to interact with target sebocytes without violating the bloodstream.

Acting as a medium through which retinoids may penetrate the stratum corneum, this invention's hydrophobic composition effectively reduces the skin's sebum output from initial application and continues to reduce sebocyte activity for a significant period of time following each application. It serves as a prophylactic for mitigating numerous health disorders, including acne, and its oil-based film protects skin from the excessive dryness, peeling and irritation commonly experienced with aforementioned skin treatments. Additional advantages inherent to the present invention are increased chemical stability of hydrophobic compositions containing retinoids, ease of use, and economical manufacture.

### DETAILED DESCRIPTION OF THE INVENTION—PREFERRED EMBODIMENTS AND OPERATION

The best mode of the present invention is prepared using methodologies well-known by an artisan of ordinary skill. The following specific embodiment is to be construed as merely illustrative and is not limited by the remainder of the disclosure in any way whatsoever.

### EXAMPLE I

The main embodiment is an oil-based composition for treating acne and oily skin. Natural Vitamin A in the form of a retinol, specifically retinyl palmitate, serves as the benefit retinoid agent. Synthesis of the preferred composition is achieved by mixing retinyl palmitate with several oils. The approximate weight percentages of these ingredients are listed below in

TABLE I

| Ingredients | % (by weight) |
|---|---|
| Sunflower Oil | 46.0 |
| Olive Oil | 26.0 |
| Clove Oil | 4.7 |
| Vitamin E | 0.3 |
| Retinyl Palmitate | 23.0 |

For purposes of treating acne, the retinoid, preferably retinyl palmitate, has been added to the composition at about 23% by weight. However, the subject invention may comprise a benefit retinoid or any combination of retinoids from about 0.00001% to about 100% by weight of the composition, but more preferably from about 10% to about 30%, and most preferably from about 20% to about 25%.

The central feature of this invention is its oleaginous quality. The “base-oils,” sunflower and olive, listed in Table I act as a foundation for the topical vehicle through which the retinoid can interact with skin. The best mode comprises about 72% base-oils; however, the subject invention may comprise a base-oil or any combination of base oils from about 0% to about 100% by weight of the composition, but more preferably from about 40% to about 90%, and most preferably from about 60% to about 85%.

Vitamin E is not absolutely necessary for operation but serves here as an antioxidant addition to protect the retinoid component. Clove oil is also not strictly necessary for operation but acts here both as an antiseptic additive and for attributing a bad taste to the composition meant to discourage accidental or purposeful ingestion. All ingredients are mixed at room temperature until homogeneous, and the resulting composition should be promptly packaged in a nonmetal container (e.g., into a polyethylene bottle) to discourage oxidation and degradation of the Vitamin A molecule.

The invention is administered topically to a biological subject, i.e., by the direct laying on or spreading of the composition on the skin of the subject (e.g., daily before retiring). The product may be applied with the fingers by lightly massaging the composition into the skin. Typically the amount required per facial application will be about 1.0 mls (20 drops), but the actual volume depends entirely on the skin area to be coated. Several applications may be applied in one 24 hour period, and the applications may be applied on top of each other in succession. The composition is preferably applied from about four times a day to about once every three days, more preferably from about twice a day to once every other day.

Immediate reduction in sebaceous gland activity may occur after the initial application. However, satisfactory resolution of acne may require chronic application for weeks, months, or years, or for any amount time required for the composition to shrink sebocytes substantially, open comedones, cure lesions, and excise scars and cysts. Upon achieving significant improvement in skin complexion, a user may proceed to use the invention indefinitely for safely maintaining healthy skin via henceforth continuing to keep sebum production in check.

### DETAILED DESCRIPTION OF THE INVENTION—ALTERNATIVE AND ADDITIONAL EMBODIMENTS

The following examples further describe and demonstrate additional and alternate embodiments of the subject invention. The examples are given solely for the purpose of illustration and are not to be construed as limitations of the subject invention, as many variations thereof are possible without departing from the spirit and scope of the invention.

The inventive composition contains, as an essential ingredient, a compound selected from the group consisting of retinoids. These compounds are well-known in the art and are commercially available from a number of sources, and one or more such retinoids may be used in combination herein. Available retinoids for use may be synthetic, derived from a substantially pure material, or be an extract obtained by suitable physical and/or chemical isolation from natural (e.g., plant) sources.

Esters of retinol, selected from retinyl palmitate, retinyl acetate and retinyl propionate, are the preferred benefit agents for use in the present invention, as these are the most commercially available, inexpensive, and efficacious retinoids for topical usage. Examples of retinyl esters may include, but are not limited to, retinyl formate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl laurate, retinyl tridecanoate, retinyl myristate, retinyl pentadecanoate, retinyl heptadeconoate, retinyl stearate, retinyl isostearate, retinyl nonadecanoate, retinyl arachidonate, retinyl behenate, retinyl linoleate, and retinyl oleate. Alternatively, the retinoid component may consist of one or more of the following isomers of retinol: all-trans-retinol, 13-cis-retinol, 11-cis-retinol, 9-cis-retinol, 3,4-didehydro-retinol. Most preferred of this class is all-transretinol, due to its wide commercial availability.

The main embodiment utilizes Vitamin E (tocopherol) as an antioxidant; however, alternative embodiments may exclude an antioxidant entirely or may employ others alone in combination. Some examples of antioxidants suitable for use in the present invention are ascorbic acid, butylated hydroxyanisole (BHA), butylated hydroxytoluene (BHT), preservatives (e.g., parabens such as methylparaben and propylparaben), and/or chelating agents (e.g., such as EDTA and edetate disodium).

Alternative embodiments of the present invention may further comprise a wide number of optional components provided that such additives are physically and chemically compatible with essential components described herein, and do not unduly impair stability, efficacy, or other use benefits associated with the compositions of the present invention. Classifications herein are made for the sake of convenience and are not intended to limit additional active ingredients to one particular application or applications listed. Optional components may include ingredients used conventionally in the art of skin care compositions, such as, but not limited to, preservatives, cosmetic preservative enhancers, and medicinally active agents in addition to the primary retinoid active.

Other optional components might include dermatologically active agents supplying cosmetic or therapeutic effects on the skin, as selected from the groups consisting of hydroxy acids, benzoyl peroxide, sulfur resorcinol, ascorbic acid, D-panthenol, hydroquinone, sunscreen agents, anti-inflammatory agents, skin lightening agents, antimicrobial agents, antifungal agents, estrogens, or mixtures thereof. Alternative embodiments may further comprise aesthetic agents, absorbents, abrasives, anticaking agents, antifoaming agents, antimicrobial agents, binders, biological additives, buffering agents, bulking agents, chemical additives, cosmetic biocides, denaturants, cosmetic astringents, drug astringents, external analgesics, film formers, humectants, opaciiying agents, fragrances, pigments, colorings, essential oils, skin sensates, emollients, skin soothing agents, skin healing agents, pH adjusters, plasticizers, preservative enhancers, propellants, reducing agents, skin-conditioning agents, skin penetration enhancing agents, skin protectants, solvents, suspending agents, emulsifiers, thickening agents, solubilizing agents, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents, exfoliants, organic hydroxy acids, vitamins and derivatives thereof, natural extracts, and any mixture thereof.

The main embodiment is non-polar and comprised substantial of oils (non-ionic lipids), as these provide the best vehicle for transporting fat-soluble retinoids. Since hydrophobicity is of central importance to the functioning of this invention, at least one constituent selected from the group consisting of oils should be present in most alternative embodiments. Non-ionic lipids may comprise from about 0% to about 100% by weight of the composition, preferably greater than 50% by weight of the composition. Though the main embodiment is comprised of base-oils derived from olive and sunflower, any number of natural or synthetic oils may be substituted or added to the composition (e.g., macadamia oil, soybean oil, rapeseed oil, clove oil, anise oil, jojoba oil, etc.).

The oil component may be aryl, aliphatic, cycloaliphatic, saturated, unsaturated, straight, branched, monounsaturated, or polyunsaturated, or may include any combination of non-ionic lipids selected from the group consisting of fatty acids, fatty alcohols, and fatty acid esters, wherein the fatty acid moiety has from about 12 to about 20 carbon atoms. Some suitable polyunsaturated fatty acids include linolenic acid, alpha-linolenic acid, alpha-linoleic acid, gamma linolenic acid, dihomo-gamma-linolenic acid, docosahexaenoic acid, eicosapentaenoic acid, neem oil, and lemon oil. Other oils available to the present invention include glyceryl monoesters, glyceryl diesters, alkoxylated alcohols, alkoxylated alkyl phenols, alkoxylated acids, alkoxylated amides, alkoxylated sugar derivatives, alkoxylated derivatives of natural oils or waxes, polyoxyethylene, polyoxypropylene block copolymers, polyoxyethylene ether fatty acids, steroids, fatty acid esters of alcohols, or any combination thereof.

### CONCLUSIONS, RAMIFICATIONS, AND SCOPE

Accordingly, the reader will see that the oil-based composition of this invention can provide a unique, convenient means for topically delivering retinoids with minimal skin irritation. The present invention offers an effective therapeutic method for abating excess sebum production and treats a multitude of skin disorders, including acne vulgaris, preadolescent acne, rosacea, premenstrual acne, acne venenata, acne cosmetica, acne detergicans, acne cosmetica, acne excoriee, gram-negative acne, steroid acne, acne conglobata, and nodulocystic acne. The present invention may be applied toward mitigating other types of acneiform dermal disorders; such as perioral dermatitis, seborrheic dermatitis in the presence of acne, negative folliculitis, sebaceous gland dysfunction, hiddradenitis suppurativa, pseudo-folliculitis barbae, and folliculitis.

The present invention may also treat skin disorders other than acne and its analogues. Topical compositions containing retinoids have been found useful in countering the physical effects of aging, depigmenting the skin, and in the prevention and treatment of melanoma and other cancers of the epidermis. The composition described herein may be used to treat other ailments of the skin, such as skin lesions, ocular disorders, wounds, ulcers, burns, radiation burns, diaper rash, blisters, psoriasis, athlete's foot, and warts. The target aliment may be found on the skin's surface, just below a superficial layer of skin, in underlying dermal tissue, in deep wounds, ulcers, or unseen by the naked eye.

Since compositions of the present invention control sebum production, they may also be used to regulate scalp oiliness, control dandruff, and treat hair loss, known as androgenetic alopecia “AGA,” which is the most common cause of hair loss in both men and women and is identifiable by the loss of hair over the vertex of the scalp.

1. A composition comprising:
(a) a vehicle selected from the group consisting of oils, and
(b) a benefit agent selected from the group consisting of retinoids, whereby topical means for treating aliments of mammalian skin will be provided.

2. The composition of claim 1 wherein said vehicle comprises a plurality selected from the group consisting of oils.

3. The composition of claim 1 wherein said benefit agent comprises a plurality selected from the group consisting of retinoids.

4. The composition of claim 1 wherein said composition is used to formulate another composition providing topical means for treating aliments of mammalian skin.

5. The composition of claim 1 wherein said aliments of mammalian skin is attributable to acne, cysts, oily skin, blackheads, whiteheads, dry skin, age spots, atopic dermatitis, non-atopic dermatitis, conjunctivitis, eczema, psoriasis, infectious disorders, wrinkles, or acne scars.

6. The composition of claim 1 formulated as a liquid, gel, cream, emulsion, suspension, foam, cream, ointment, or stick.

7. The composition of claim 1 wherein said benefit agent comprises one or more retinoids selected from the group consisting retinol, retinal, and retinyl esters, all-trans retinoic acid, or 13-cis-retinoic acid.

8. The composition of claim 1 wherein said benefit agent comprises one or more esters selected from the group consisting of retinyl palmitate, retinyl propionate, retinyl acetate, retinyl butyrate, retinyl octanoate, retinyl laurate, retinyl oleate and retinyl linoleate.

9. The composition of claim 1 further comprising one or more selected from the group consisting of vitamins, botanical extracts, exfoliants, peeling agents, skin renewal agents, carboxylic acids, keto acids, α-hydroxy acids, β-hydroxy acids, retinoids, peroxides, organic alcohols, sunscreen, tanning agents, sunburn treatment products, cleansers, astringents, toners, rinses, serums, masks, cosmetic products, antibiotics, analgesics, creams, lotions, moisturizers, soaps, detergents, acne treatment agents, or sebum reducing agents.

10. A composition comprising:
(a) a vehicle selected from the group consisting of oils, and
(b) a benefit agent selected from the group consisting of retinoids, whereby topical means for treating aliments of the hair will be provided.

11. The composition of claim 10 wherein said aliments of the hair is attributable to hair loss.

12. The composition of claim 10 wherein said benefit agent comprises one or more retinoids selected from the group consisting retinol, retinal, and retinyl esters, all-trans retinoic acid, or 13-cis-retinoic acid.

13. The composition of claim 10 wherein said benefit agent comprises one or more esters selected from the group consisting of retinyl palmitate, retinyl propionate, retinyl acetate, retinyl butyrate, retinyl octanoate, retinyl laurate, retinyl oleate and retinyl linoleate.

14. The composition of claim 10 formulated as a liquid, gel, cream, emulsion, suspension, foam, cream, ointment, or stick.

15. The composition of claim 10 wherein said vehicle comprises a plurality selected from the group consisting of oils.

16. The composition of claim 10 wherein said benefit agent comprises a plurality selected from the group consisting of retinoids.

17. The composition of claim 10 wherein said composition is used to formulate another composition providing topical means for treating aliments of the hair.

18. The composition of claim 10 further comprising one or more benefit agents selected from the group consisting of depilatory agents, bracers, aftershave products, shampoos, conditioners, colorants, dyes, bleaching agents, anti-dandruff agents, permanent wave agents, hair straightening agents, or hair treatment products.

19. A composition comprising:
(a) a vehicle selected from the group consisting of oils, and
(b) a benefit agent selected from the group consisting of retinoids, whereby topical means for treating aliments of the eye will be provided.
